(19) United States
(12) Patent Application Publication
(10) Pub. No.: US 2022/0008695 A1
(43) Pub. Date: Jan. 13, 2022

(54) MEDICAL CONDUIT SECURING DEVICE

(71) Applicant: Dale Gatti, Pompano Beach, FL (US)

(72) Inventor: Dale Gatti, Pompano Beach, FL (US)

(21) Appl. No.: 17/369,254

(22) Filed: Jul. 7, 2021

Related U.S. Application Data
(60) Provisional application No. 63/048,911, filed on Jul. 7, 2020.

(51) Int. Cl. A61M 25/02

(52) U.S. Cl. CPC A61M 25/02; A61M 2025/026

(57) ABSTRACT

A medical conduit securing device is disclosed. The medical conduit securing device includes a first end; a second end opposing the first end, wherein the second end defines a flexible aperture; a flexible elongated body spanning a distance between the first end and the second end, the body having a plurality of raised areas; wherein reception of the first end through the second end via the flexible aperture cooperates to define a space shaped and sized to ensnare at least one medical conduit. The medical conduit securing device is configured to be attached to garments or ornaments donned by the user is a sanitary manner that does not abrase the medical conduits.

### CROSS-REFERENCE TO RELATED APPLICATION

This application claims priority to U.S. Provisional Patent Application No. 63/048,911 filed Jul. 7, 2020, the entirety of which is incorporated by reference.

### FIELD OF THE INVENTION

The present invention relates generally to securing medical tubing, and, more particularly, relates to a device that secures medical conduits attached to fluid ports in the body of the patient.

### BACKGROUND

While an individual is receiving certain medical treatments, there can be a plethora of conduits and/or tubes connected/attached to the individual such as but not limited to IV tubing, catheter tubing, tubing associated with a port for cancer patients, and multiple other tubing mechanisms for delivering and extracting applicable fluids and substances from the patient. In order to prevent consistent placement or unintended removal of the aforementioned tubes, the standard in the medical industry is to secure the tubes together via a bundling mechanism such as tape. The bundling of the tubes creates a load or weight in which ordinarily the patient is tasked with bearing. A major drawback to bundling mechanisms like tape is that it sticks and is hard to remove. The retention strength of the tape is not modifiable; therefore, requiring the medical professional to cut the tape with scissors or a knife and utilize new tape each time the conduits need to be bundled. A major drawback to this approach is that the scissor or knife's sharp point can penetrate the one or more tubes in the bundle. A puncture endured by the one or more tubes significantly increases the possibility of infection, which can be fatal in instances when the patient has a weakened or compromised immune system (i.e., a cancer patient). The cut can also compromise the flow of critical substances to and from the patient. In addition, when the tape becomes wet, it quickly becomes microbial, rendering it a hotbed for germs and bacteria. Furthermore, a solvent is required to remove the tape's glue from the lines. The solvent, if introduced into the lines by, for example, a hole in the line, could be fatal to the patient.

Another common issue with the bundling of medical conduits to a patient is the securing of the bundle to the patient. One current approach is to attach the bundle of tubes directly to garment donned by the patient via a safety pin allowing the bundle of tubes to suspend from the garment. However, a major drawback to this approach is that the safety pin's sharp point can penetrate the one or more tubes in the bundle compromising the flow of substances to and from the patient and/or introducing bacteria to the line.

Furthermore, it is common for patients to have to disconnect the bundle of tubing from their garment, particularly in situations where the patient is changing garments or when the patient has to wash their body. The tape and safety pin is less than ideal for this. The patient is required to reattach the bundle to their clothing via puncturing the safety pin through their garment resulting in multiple holes in the garment. In addition, the load or weight of the bundled tubes consistently pulling on the safety pin results in damage to the garment via stretching or tearing of the garment.

Therefore, there exists a need for a mechanism to bundle medical conduits and securely attach them to a patient in a manner that circumvents the aforementioned issues associated with the current mechanisms and practices.

### SUMMARY OF THE DISCLOSURE

The invention provides a medical conduit securing device and a method for securing a medical conduit to a patient that overcomes the hereinafore-mentioned disadvantages of the heretofore-known devices and methods of this general type and that effectively facilitates safely bundling at least one medical conduit and securing the at least one medical conduit to the patient.

Although the invention is illustrated and described herein as embodied in a medical conduit securing device, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

With the foregoing and other objects in view, there is provided, in accordance with the invention, a medical conduit securing device that includes a first end; a second end opposing the first end, wherein the second end defines a flexible aperture; a flexible elongated body spanning a distance between the first end and the second end, the body having a plurality of raised areas; wherein reception of the first end through the second end via the flexible aperture cooperates to define a space shaped and sized to ensnare at least one medical conduit.

In accordance with a further feature of the present invention, a slot is disposed on the second end designed and configured to receive at least a portion of a retaining mechanism. The retaining mechanism is configured to be affixed to an object or garment donned by the patient.

In accordance with a further feature of the present invention, the flexible elongated body is composed of an antimicrobial material.

In accordance with a further feature of the present invention, the plurality of raised areas is defined by a series of incrementally increasing sizes of the raised areas, each raised area including a dimension greater than the preceding raised area and each dimension greater than the flexible aperture.

In accordance with a further feature of the present invention, the at least the flexible elongated body is configured to be composed of material devoid of one or more sharp edges

With the foregoing and other objects in view, there is provided, in accordance with the invention, a method of securing a medical conduit to a patient. The method including providing a flexible elongated body spanning a distance between a first end and a second end; wherein the flexible elongated body includes a plurality of raised areas and the second end includes a flexible aperture; positioning at least one medical conduit between the first and second ends allowing the medical conduit to be in contact with the flexible elongated body; inserting the first end through the flexible aperture; wherein the flexible aperture is configured to be proximate to at least one raised area of the plurality of raised areas cooperating to define a space sized to ensnare the at least one medical conduit; retaining the at least one medical conduit with the defined space; and affixing the flexible elongated body to an object or garment donned by a patient.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of a medical conduit securing device, according to an example embodiment;

FIG. 2 is an elevational side view of the medical conduit securing device of FIG. 1, according to an example embodiment; and

FIG. 3 is a perspective view of the medical conduit securing device of FIG. 1 ensnaring a plurality of medical conduits, according to an example embodiment; and

FIG. 4 is a flow chart diagram illustrating an exemplary method for securing a medical conduit to a patient, according to an example embodiment.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient medical conduit securing device configured to ensnare or bundle one or more medical conduits in a manner that allows the bundled medical conduits to be attached to a patient in a secure, comfortable, and sanitary manner. The medical conduit securing device includes a first end; a second end opposing the first end, wherein the second end defines a flexible aperture; and a flexible elongated body spanning a distance between the first end and the second end, the body having a plurality of raised areas; wherein reception of the first end through the second end via the flexible aperture cooperates to define a space shaped and sized to ensnare at least one medical conduit. The ensnaring and/or bundling of the one or more medical conduits is performed in a manner in which the medical conduits are not subject to puncturing, rupturing, or undesired mobility. In addition, a retaining mechanism is provided configured to be received by the medical conduit securing device in order to attach to an object or garment donned by the patient.

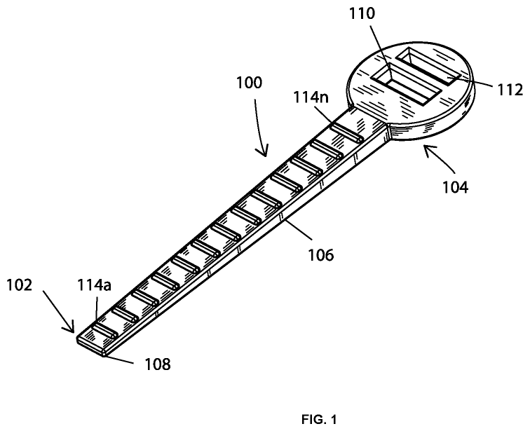

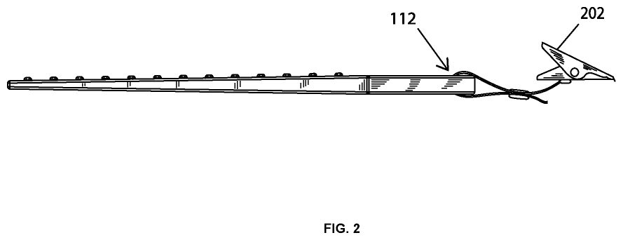

Referring now to FIG. 1, a medical conduit securing device 100 is depicted, according to an exemplary embodiment. In some embodiments, medical conduit securing device 100 includes a first end 102, a second end 104, and a flexible elongated body 106 configured to span a distance from first end 102 to second end 104. A first side of the flexible elongated body 106 is visible in FIG. 1. A second side of flexible elongated body 106 (not visible in FIG. 2) longitudinally opposes the first side. The first end 102 can be brought into contact with the second end 104 only by a user-applied force or movement applied to at least one of the first end 102 or the second end 104. It is to be understood that the movement or force applied by the user is one of a bending movement allowing the first end 102 and the second end 104 to move closer to each other. Flexible elongated body 106 may be coated and/or composed of an antimicrobial material. Antimicrobial materials include but are not limited to graphene, biopolymer, carbon nanotubes, magnesium oxide, titanium dioxide nanoparticles, or any other applicable material configured to destroy microorganism or prevent their growth. In some embodiments, the flexible elongated body 106 may be made of materials that are devoid of sharp edges to prevent abrasion of retained medical conduits; thus, the conduits are not subjected to a high probability of puncturing or damage. In some embodiments, the first end 102, the second end 104, and/or the flexible elongated body 106 include a covering or wrapping, e.g., decorative sleeves, which are intended to provide customized aesthetics to the medical conduit securing device 100. It is to be understood that various shapes, sizes, lengths, and dimensions of medical conduit securing device 100 are within the scope and spirit of the disclosure.

In some embodiments, first end 102 includes a latching mechanism 108 configured to be inserted through second end 104, in which second end 104 includes a flexible aperture 110 configured to be penetrated via the latching mechanism 108. In the embodiment shown, the latching mechanism 108 is a raised area that renders an overall dimension of the first end 102 larger than a natural resting dimension of the flexible aperture 110. When the first end 102 is inserted within the flexible aperture 110, the flexible aperture 110 is deformed from its natural resting shape. Once the latching mechanism 108 moves inside the flexible aperture 110, the flexible aperture returns to it natural resting shape and the latching mechanism 108 prevents the first end from being removed unless and until a particular amount of pulling force is applied. Pulling force is effective in removing the first end 102 from the aperture 110 because the aperture 110, the latching mechanism 108, or both is flexible, i.e., deformable.

In some embodiments, latching mechanism 108 serves as an introductory mechanism for flexible aperture 110 to be exposed to a plurality of raised areas 114a-114n, in which n represents any number larger than a. It is to be understood that the value of a-n is subject to the length of flexible elongated body 106, in which raised areas 114a-114n are configured to be equally spaced from each other along at least one surface (top or bottom) of flexible elongated body 106, and in some embodiments each of raised areas 114a-114n incrementally increase in size with the dimension of each greater than the dimensions of flexible aperture 110. The greater the length of flexible elongated body 106, the larger number n is. In a preferred embodiment, raised areas 114a-114n are allocated along a top surface of flexible elongated body 106 as illustrated in FIG. 1. The flexible build of flexible elongated body 106 in combination with flexible aperture 110 allow the plurality of raised areas 114a-114n to be actively passed through flexible aperture 110 while still maintaining a tight retention to the conduits that flexible elongated body 106 is in contact with.

In some embodiments, the medical conduit securing device 100 includes a slot 112 configured to support attachment of the medical conduit securing device 100 to another object or garment. It is to be understood that, in a preferred embodiment, slot 112 is allocated proximate to flexible aperture 110; however, slot 112 may be allocated in any applicable location of medical conduit securing device 100 that allows medical conduit securing device 100 to be donned by the user.

Referring now to FIG. 2, an elevational side view of the medical conduit securing device 100, including a retaining mechanism 202 being received or inserted through slot 112, is depicted, according to an exemplary embodiment. It is to be understood that retaining mechanism 202 may be associated with a clip, lanyard, key holder, badge holder, carabiner, or any other applicable mechanism configured to be attached to an object and/or ornament configured to be donned by a user in a manner that does not unnecessarily tug/pull or cause discomfort. The purpose of the configuration of retaining mechanism 202 is to ensure that medical conduit securing device 100 does not tug or pull on the object or ornament retaining mechanism 202 is attached to when medical conduit securing device 100 is simultaneously retaining the coupling and bearing the weight. In some embodiments, retaining mechanism 202 may be configured to be attached to a necklace, loop, or any other applicable ornament configured to be donned by the user known to those of ordinary skill in the art.

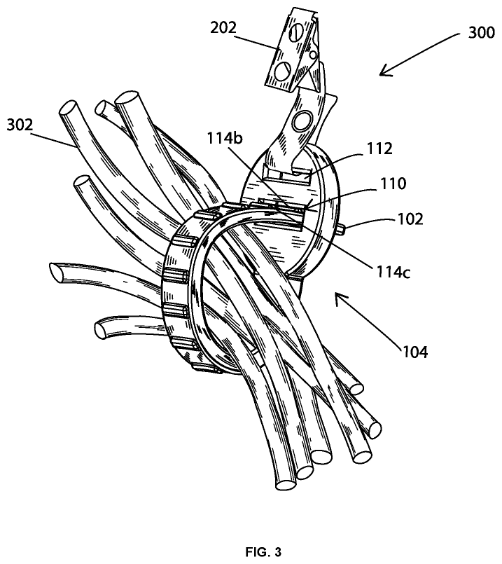

Referring now to FIG. 3, a prospective view of medical conduit securing device 100 ensnaring a plurality of medical conduits 302 is depicted as assembly 300, according to an exemplary embodiment. It is to be understood that, although medical conduit securing device 100 is configured to ensnare medical conduits, medical conduit securing device 100 may also be configured to ensnare wiring, tubing, hoses, or any other applicable type of capillary-like structure. In some embodiments, first end 102 is inserted through flexible aperture 110 allowing at least one of plurality of raised areas 114a-114n to come into contact with flexible aperture 110. First end 102 and second end 104 cooperate to define an area shaped and sized to ensnare at least one medical conduit. However, in the instance where the area needs to be adjusted to accommodate plurality of medical conduits 302 (i.e., tightening the retention of medical conduit securing device 100), the user may pull on first end 102 resulting in the current raised area (i.e., 114b) occupying flexible aperture 110 to be forced through flexible aperture 110 allowing the subsequent raised area (i.e., 114c) of the plurality of raised areas to be in contact with flexible aperture 110. Contact between at least one of the plurality of raised areas 114a-114n and flexible aperture 110 may be accomplished in a variety of manners subject to the configuration of medical conduit securing device 100. For example, flexible aperture 110 and at least one of plurality of raised areas 114a-114n may support an interlocking coupling, a magnetic-based coupling, a width/dimension-based coupling (in which a particular orientation of plurality of raised areas 114a-114n may render occupation of space within flexible aperture 110), or any other applicable coupling and/or affixing combination configured to securely ensnare plurality of medical conduits 302 without damaging them or unnecessarily increasing the load to be sustained by retaining mechanism 202. It is to be understood that as each raised area of plurality of raised areas 114a-114n is being passed through flexible aperture 110, the area shaped and sized to ensnare at least one medical conduit becomes smaller; thus, increasing retention force between flexible elongated body 106 and plurality of medical conduits 302.

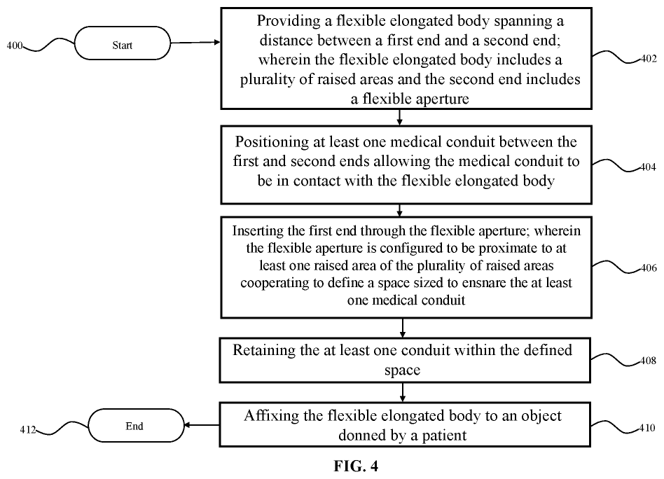

Referring now to FIG. 4, a method of securing a medical conduit 400 is depicted, according to an exemplary embodiment. It is to be understood that the method is limited to securing a medical conduit to a patient, and other environments that require tethering and attachment are within the spirit and scope of the disclosure. At step 400, the method begins in which a user and/or patient wishes to bundle at least one medical conduit for attachment to an object associated with the user. In some embodiments, the object may be a collar, shirt pocket, sleeve, belt, or any other graspable component donned by a user. At step 402, flexible elongated body 106 is provided in which a distance extending between first end 102 and second end 104 is configured to be in proximate and/or direct contact with at least one medical conduit. At step 404, the user positions the one or more medical conduits between first end 102 and second end 104. In some embodiments, the one or more medical conduits are configured to be aligned in a manner that is perpendicular to flexible elongated body 106. In some embodiments, once the at least one medical conduit is in contact with flexible elongated body 106, at least one of first end 102 and/or second end 104 may receive a bending force to initiate the contact between first end 102 and second end 104 necessary in order to form the space sized to ensnare the at least one medical conduit. At step 406, the user applies the bending force to first end 102 allowing latching mechanism 108 to be inserted through flexible aperture 110. As a result of latching mechanism 108 being inserted through flexible aperture 110, at least one of plurality of raised areas 114a-114n are presented in contact with flexible aperture 110. At step 408, the contact between at least one of plurality of raised areas 114a-114n and flexible aperture 110 retains medical conduits 302 within the space defined by first end 102 being in contact with second end 104. It is to be understood that the size of the defined space designated for medical conduits 302 is determined based on which of raised areas 114a-114n is in contact with flexible aperture 110. The higher the value of n, the greater the retention force applied to medical conduits 302. At step 410, once medical conduits 302 are firmly bundled together by the retention force desired by the user, medical conduit securing device 100 is secured to a garment or object donned by the user via retaining mechanism 202, which is used for affixing, attaching, or clamping the assembly 300 to the garment. In some embodiments, retaining mechanism 202 is configured to suspend from the garment allowing enough slack for medical conduit securing device 100 to suspend from, but without snagging on the user's garment; thus, preventing puncturing or tearing of the garment that device 100 is attached to. At step 412, the method ends. Because the aperture 110 and/or the retaining mechanisms 114a-n (or both) are flexible/deformable, the steps FIG. 4 can be performed in reverse just as easily as they can be in the order shown in FIG. 4. This provides a significant advantage over prior-art medical-conduit-retaining methods and mechanisms, which lock or adhere once attached and are not removable without destroying the retaining mechanism. The present invention easily attaches and detaches as needed and without limitation. It does not require bringing sharp objects near the fluid conduits and it does not damage the clothing of the patient.

What is claimed is:

1. A reusable medical conduit securing device comprising:
a first end;
a second end opposing the first end, the second end defining:
a flexible aperture; and
a portion configured to couple to a retaining mechanism;
a flexible elongated body spanning a distance between the first end and the second end, the body having a plurality of raised areas where a dimension of the body with raised areas exceeds a dimension of the natural resting shape of the flexible aperture;
wherein reception of the first end through the second end via the flexible aperture cooperates to define a space shaped and sized to ensnare at least one medical conduit; and
wherein the flexible aperture allows for insertion and removal of first end of the medical conduit securing device into and out of the flexible aperture without permanent alteration of the medical conduit securing device.

2. The reusable medical conduit securing device of claim 1, wherein the retaining mechanism is configured to be affixed to an object donned by the user.

3. The reusable medical conduit securing device of claim 1, wherein the flexible elongated body includes antimicrobial material.

4. The reusable medical conduit securing device of claim 1, wherein the plurality of raised areas is defined by a series of incrementally increasing sizes of the raised areas.

5. The reusable medical conduit securing device of claim 1, wherein at least the flexible elongated body is devoid of sharp edges.

6. A method of securing a reusable medical conduit to a patient, the method comprising:
providing a flexible elongated body spanning a distance between a first end and a second end;
wherein the flexible elongated body includes a plurality of raised areas and the second end includes a flexible aperture;
positioning at least one medical conduit between the first and second ends allowing the medical conduit to be in contact with the flexible elongated body;
inserting the first end through the flexible aperture by applying a first force to the first end in a first direction until at least one of the plurality of raised areas passes through the flexible aperture;
affixing the flexible elongated body to an object donned by a patient; and
removing the least one of the plurality of raised areas from the flexible aperture by applying a second force to the first end that is approximately equal to the first force, but in a second direction that is opposite the first direction.

7. The method of claim 6, wherein affixing the flexible elongated body includes receiving at least a portion of a retaining mechanism through a slot disposed on the second end.

8. The method of claim 6, wherein the plurality of raised areas is defined by a series of incrementally increasing sizes of the raised areas.

9. The method of claim 6, wherein the flexible elongated body includes antimicrobial material.

10. The method of claim 6, wherein the flexible elongated body is devoid of sharp edges.
